(19) United States
(12) Patent Application Publication
(10) Pub. No.: US 2025/0263740 A1
(43) Pub. Date: Aug. 21, 2025

(54) NON-REPLICATING BOVINE INFECTIOUS LYMPHOMA VIRUS (BLV) AND CELLS FOR PRODUCING SAME

(71) Applicant: THE UNIVERSITY OF TOKYO, Tokyo (JP)

(72) Inventors: Yoko AIDA, Tokyo (JP); Samy Gamal Alsaied Mohamed METWALLY, Tokyo (JP); Ryosuke MATSUURA, Tokyo (JP)

(21) Appl. No.: 18/857,565

(22) PCT Filed: Apr. 19, 2023
(86) PCT No.: PCT/JP2023/015633

§ 371 Date: Mar. 14, 2025

(30) Foreign Application Priority Data
Apr. 20, 2022 (JP) 2022-069710

(51) Int. Cl. C12N 15/86; A61K 39/00; A61K 39/21

(52) U.S. Cl. CPC C12N 15/86; A61K 39/21; A61K 2039/5254; C12N 2740/14021; C12N 2740/14034; C12N 2740/14043

(57) ABSTRACT

An object of the present invention is to provide a novel non-replicating bovine leukemia virus (BLV) and a producing cell thereof. According to the present invention, there is provided a bovine leukemia virus (BLV) in which at least a part of the function of a pol gene is deficient. Also, according to the present invention, there is provided a non-replicating BLV-producing cell comprising a gene of a BLV in which at least a part of the function of a pol gene is deficient. The present invention is advantageous in that it can provide a BLV vaccine which is highly immunogenic, and is highly safe without replicating in an infected subject.

### CROSS-REFERENCE TO RELATED APPLICATION

The present application enjoys the benefit of priority from the prior Japanese Patent Application No. 2022-69710 filed on Apr. 20, 2022, the entire disclosure of which is incorporated herein by reference.

### TECHNICAL FIELD

The present invention relates to a non-replicating bovine leukemia virus (BLV) and a producing cell thereof.

### BACKGROUND ART

The bovine leukemia virus (BLV) is a causative virus of enzootic bovine leukosis (EBL) which is a malignant B lymphoma, and is a retrovirus which is integrated as a provirus into the DNA of a host cell. About 70% of BLV-infected cattle remain presymptomatic and healthy, while about 30% thereof have persistent lymphocytosis, and, after a long period of latency, about a few percent thereof develop bovine leukosis (Non-patent Documents 1 and 2).

Although EBL has been increasing in recent years, a vaccine effective in preventing EBL has not yet been developed. One of the main factors for this is that the amount of the virus to be produced from BLV-infected cells is extremely small, about 1/1,000 to 1/10,000 of the amount of AIDS virus to be produced, which belongs to the same Retrovirus family, thereby making it difficult to develop an inactivated vaccine or a cell-derived vaccine. Attempts have been made so far, for example, to develop expression vectors for increasing the amount of BLV to be produced (Patent Document 1). However, there is a risk that, after infection with BLV which is a retrovirus, the viral gene will be integrated into the genome of a host, and therefore problems with practical use of a live vaccine still remain.

### REFERENCE LIST

### Patent Documents

Patent Document 1: JP 2019-24351 A

### Non-Patent Documents

Non-Patent Document 1: Gillet NA, et al., Retrovirology, 2007, 4:18.
Non-Patent Document 2: Aida Y, et al., Frontiers in Microbiology, 2013, 4:328.

### SUMMARY OF THE INVENTION

An object of the present invention is to provide a novel non-replicating bovine leukemia virus (BLV) and a producing cell thereof.

The present inventors have now succeeded in establishing a virus-producing cell line that produces an infectious but replication-incompetent bovine leukemia virus (BLV). As a result of analysis of the gene sequence of the virus produced by the cell line, the present inventors have also found that a part of the pol gene is deleted. Further, the present inventors have found that a cell having introduced thereinto a BLV gene in which a part of the pol gene is deleted (pBLV-416ΔRT-introduced cell) has increased expression level and release amount of the virus produced and also has enhanced syncytium formation ability, as compared with a control cell (pBLV-416-introduced cell). The present inventors have also found that, upon inoculation of mice with the virus, an anti-p24 antibody increased over time, whereas no BLV gene was detected. As a result of studies using a non-replicating BLV-producing cell line (PK15-BLVΔRT cell line), the present inventors have also found that the virus produced from the cell line can be useful as a non-replicating BLV vaccine. The present invention is based on the findings.

According to the present invention, the following inventions are provided.
[1] A bovine leukemia virus (BLV) in which at least a part of the function of a pol gene is deficient.
[2] The virus according to [1], wherein the function of the pol gene is the function of a reverse transcriptase and/or an integrase.
[3] The virus according to [1] or [2], wherein at least a part of the pol gene is mutated.
[4] The virus according to [3], wherein the mutation is a deletion of 1 to 1643 bases in a reverse transcriptase region of the pol gene and/or 1 to 894 bases in an integrase region of the pol gene.
[5] The virus according to [3], wherein the mutation is a mutation in bases corresponding to 1^{st} to 1643^{rd} bases and/or bases corresponding to 1644^{th} to 2537^{th} bases from the 5′ side in a base sequence of the pol gene as shown in SEQ ID NO: 2.
[6] The virus according to any one of [1] to [5], which does not replicate in an infected subject.
[7] A non-replicating bovine leukemia virus (BLV)-producing cell, comprising a gene of a BLV in which at least a part of the function of a pol gene is deficient.
[8] A method for producing a non-replicating BLV, comprising the step of culturing the cell according to [7].
[9] A BLV vaccine comprising the virus according to any one of [1] to [6].
[10] A method for preventing or treating a BLV, comprising the step of inoculating a subject excluding a human with the virus according to any one of [1] to [6] or the vaccine according to [9].
[11] A method for producing a non-replicating bovine leukemia virus (BLV)-producing cell, comprising the step of introducing, into a host cell, a gene of a BLV in which at least a part of the function of a pol gene is deficient.

The present invention is advantageous in that it can provide a bovine leukemia virus (BLV) vaccine which is highly immunogenic, and is highly safe without replicating in an infected subject.

### BRIEF DESCRIPTION OF THE DRAWINGS

FIG. 1 shows the genetic constitution of a wild-type BLV, and shows a deleted portion of a pol gene of a CMVΔU3-pBLV-416ΔRT plasmid (pBLV-416ΔRT) in comparison with a CMVΔU3-pBLV-416 plasmid (pBLV-416).

FIG. 2A shows results of PCR amplification of pol gene regions of pBLV-416ΔRT and pBLV-416, respectively. The negative control used was a pBluescript II KS(−) plasmid which was an empty vector, and the positive control used was an FLK-BLV cell which was a persistently BLV-infected cell. FIG. 2B shows results of Western blotting using a BLV-infected bovine serum (left) and a non-BLV-infected bovine serum (right) for expression of viral proteins produced by a pBLV-416ΔRT-introduced cell and a pBLV-416-introduced cell, respectively. The negative control used was a cell introduced with a pBluescript II KS(−) plasmid which was an empty vector, and the positive control used was an FLK-BLV cell which was a persistently BLV-infected cell.

FIG. 3 shows results of an immunofluorescent antibody method for intracellular localization of the viral proteins in the pBLV-416ΔRT-introduced cell and the pBLV-416-introduced cell, respectively. The negative control used was a cell introduced with a pBluescript II KS(−) plasmid which was an empty vector, and the positive control used was an FLK-BLV cell which was a persistently BLV-infected cell.

FIG. 4 shows results of Western blotting for expression levels of the viral proteins in the pBLV-416ΔRT-introduced cell and the pBLV-416-introduced cell, respectively. The negative control used was a cell introduced with a pBluescript II KS(−) plasmid which was an empty vector, and the positive control used was an FLK-BLV cell which was a persistently BLV-infected cell. As a result of a Student's t-test, * indicates p<0.05 and ** indicates p<0.01.

FIG. 5 shows results for syncytium formation ability in the pBLV-416ΔRT-introduced cell and the pBLV-416-introduced cell, respectively. The negative control used was a cell introduced with a pBluescript II KS(−) plasmid which was an empty vector. As a result of a Student's t-test, ** indicates p<0.01 and *** indicates p<0.001.

FIG. 6 shows results for cell-to-cell infection ability in the pBLV-416ΔRT-introduced cell and the pBLV-416-introduced cell, respectively. The negative control used was a cell introduced with a pBluescript II KS(−) plasmid which was an empty vector, and the positive control used was an FLK-BLV cell which was a persistently BLV-infected cell.

FIG. 7A shows release amounts of the viral protein produced by the pBLV-416ΔRT-introduced cell and the pBLV-416-introduced cell, respectively. FIG. 7B shows the reverse transcriptase activities of the viral protein produced by the pBLV-416ΔRT-introduced cell and the pBLV-416-introduced cell, respectively. FIG. 7C shows results for syncytium formation ability in the pBLV-416ΔRT-introduced cell and the pBLV-416-introduced cell, respectively. FIG. 7D shows results of Western blotting for the viral protein released from the pBLV-416ΔRT-introduced cell and the pBLV-416-introduced cell, respectively. The negative control used was a cell introduced with a pBluescript II KS(−) plasmid which was an empty vector, and the positive control used was an FLK-BLV cell which was a persistently BLV-infected cell. As a result of a Student's t-test, *** indicates p<0.001.

FIG. 8 shows the genetic constitution of a wild-type BLV, and shows a deleted portion of a pol gene of a CMVΔU3-pBLV-IFΔRT plasmid (pBLV-IFΔRT) in comparison with a CMVΔU3-pBLV-IF plasmid (pBLV-IF).

FIG. 9A shows results of Western blotting for expression levels of the viral proteins in the pBLV-IFΔRT-introduced cell and the pBLV-IF-introduced cell, respectively. FIG. 9B shows results for syncytium formation ability in the pBLV-IFΔRT-introduced cell and the pBLV-IF-introduced cell, respectively. FIG. 9C shows results of measuring amounts of the virus in culture supernatants of the pBLV-IFΔRT-introduced cell and the pBLV-IF-introduced cell, respectively, by Capture ELISA. The negative control used was a cell introduced with a pBluescript II KS(−) plasmid which was an empty vector, and the positive control used was an FLK-BLV cell which was a persistently BLV-infected cell. As a result of a Student's t-test, * indicates p<0.05, ** indicates p<0.01, and *** indicates p<0.001.

FIG. 10A shows a schedule of an experiment in which mice were inoculated with the viral protein produced by the pBLV-416ΔRT-introduced cell and the pBLV-416-introduced cell. FIG. 10B shows results of a Nested PCR method for BLV genes in blood cells 3 weeks after inoculation. FIG. 10C shows results of co-culturing a spleen cell extracted from mice and a CC81-GREMG cell. FIG. 10D shows results of quantifying anti-p24 antibodies from 0 to 6 weeks after inoculation by ELISA. The negative control used was PBS, and the positive control used was an FLK-BLV cell which was a persistently BLV-infected cell. As a result of a Student's t-test, * indicates p<0.05.

FIG. 11 shows the genetic constitution of a wild-type BLV, and shows a deleted portion of a pol gene of a BLV in a PK15-BLVΔRT cell line in comparison with a CMVΔU3-pBLV-416 plasmid (pBLV-416).

FIG. 12 shows results of Western blotting for expression levels of the viral proteins in the PK15-BLVΔRT cell line. The positive control used was an FLK-BLV cell which was a persistently BLV-infected cell.

FIG. 13 shows results of an immunofluorescent antibody method for intracellular localization of the viral protein in the PK15-BLVΔRT cell line. The positive control used was an FLK-BLV cell which was a persistently BLV-infected cell.

FIG. 14A shows a release amount of the viral protein produced by the PK15-BLVΔRT cell line. FIG. 14B shows a standard curve created using His-p24 antigen as a standard.

### DETAILED DESCRIPTION OF THE INVENTION

### Virus and Method for Producing Same

The “bovine leukemia virus (BLV)” is a causative virus of enzootic bovine leukosis (EBL), which is a malignant B lymphoma, and is a retrovirus which is integrated as a provirus into the DNA of a host cell. The genome of a wild-type BLV has a full length of about 8,720 bp, and is composed of gag, pro, pol and env genes that encode structural proteins; rex and tax genes that encode regulatory proteins; and R3 and G4 genes that encode accessory proteins, in a form in which the genes are sandwiched between two identical long terminal repeat (LTR) sequences (see FIG. 1). The gag gene encodes a protein involved in viral particle formation, the pol gene encodes a reverse transcriptase and an integrase, and the env gene encodes a coat protein involved in adsorption onto and entry into a host cell. An example of the gene sequence of the wild-type BLV is shown in Table 1 (the gene sequence of the wild-type BLV shows a sequence from the 5′ LTR to the 3′ LTR, and the underlined part shows a sequence encoding the pol gene). In the present invention, a wild-type BLV sequence other than the gene sequence indicated in Table 1 can be used.

TABLE 1
Gene sequence of wild-type BLV (SEQ ID NO: 1)

TGTATGAAAGATCATGCCGACCTAGGCGCCGCCACCGCCCCGTAAACC
AGACAGAGACGTCAGCTGCCAGAAAAGCTGGTGACGGCAGCTGGTGGC
TAGAATCCCCGTACCTCCCCAACTTCCCCTTTCCCGAAAAATCCACAC
CCTGAGCTGCTGACCTCACCTGCTGATAAACTAATAAAATGCCGGCCC
TGTCGAGTTAGCGGCACCAGAAGCGTTCTTCTCCTGAGACCCTCGTGC
TCAGCTCTCGGTCCTGAGCTCTCTTGCTCCCGAGACCTTCTGGTCGGC
TATCCGGCAGCGGTCAGGTAAGGCAAACCACGGTTTGGAGGGTGGTTC
TCGGCTGAGACCACCGCGAGCTCTATCTCCGGTCCTCTGACCGTCTCC
ACGTGGACTCTCTCCTTTGCCTCCTGACCCCGCGCTCCAAGGGCGTCT
GGCTTGCACCCGCGTTTGTTTCCTGTCTTACTTTCTGTTTCTCGCGGC
CCGCGCTCTCTCCTTCGGCGCCCACTAGCGGCCAGGAGAGACCGGCAA
ACAATTGGGGGCTCGTCCGGGATTGATCACCCCGGAACCCTAACAACT
CTCTGGACCCACCCCCTCGGCGGCATTTTGGGTCTCTCCTTCAAATTA
TATCATGGGAAATTCCCCCTCCTATAACCCCCCCGCTGGTATCTCCCC
CTCAGACTGGCTCAACCTTCTGCAAAGCGCGCAAAGGCTCAATCCGCG
ACCCTCTCCTAGCGATTTTACCGATTTAAAGAATTACATCCATTGGTT
TCATAAGACCCAGAAAAAACCATGGACTTTCACTTCTGGTGGCCCCAC
CTCATGTCCACCCGGGAGGTTCGGCCGGGTTCCCCTTGTCTTGGCCAC
CCTAAACGAAGTGCTCTCAAACGAAGGGGGCGCCCCGGGTGCATCGGC
CCCAGAAGAACAACCCCCCCCTTATGACCCCCCCGCCGTTTTGCCAAT
CATATCTGAAGGGAATCGCAACCGCCATCGTGCTTGGGCACTCCGAGA
ATTACAAGATATCAAAAAGGAAATTGAAAATAAGGCACCGGGTTCGCA
AGTATGGATACAAACACTACGACTTGCAATCCTGCAGGCCGACCCTAC
TCCGGCTGACCTAGAACAACTTTGCCAATATATTGCTTCCCCGGTCGA
CCAAACGGCCCATATGACCAGCCTAACGGCAGCAATTGCCGCCGCTGA
AGCGGCCAACACCCTCCAGGGTTTTAACCCCCAAAACGGGACCCTAAC
CCAACAATCAGCTCAGCCCAACGCCGGGGATCTTAGAAGTCAATATCA
AAACCTCTGGCTTCAGGCCTGGAAAAATCTCCCTACTCGTCCTTTAGT
ACAACCTTGGTCCACCATCGTCCAAGGCCCCGCCGAAAGCTATGTAGA
GTTTGTCAACCGGTTACAAATTTCATTAGCTGACAACCTTCCCGACGG
AGTCCCTAAGGAACCCATTATTGACTCCCTTAGTTATGCAAATGCTAA
CAAAGAGTGTCAGCAAATTTTGCAAGGGCGAGGCCTAGTGGCCGCCCC
GGTGGGGCAAAAACTGCAGGCTTGCGCACATTGGGCCCCCAAGATGAA
ACAGCCTGCAATTCTCGTCCACACCCCAGGGCCCAAGATGCCCGGGCC
TCGGCAACCGGCCCCCAAAAGGCCTCCCCCAGGACCATGCTATCGATG
CCTCAAAGAAGGCCATTGGGCCCGGGATTGTCCTACCAAGGCCACCGG
CCCCCCTCCGGGACCTTGCCCCATATGTAAAGATCCTTCCCATTGGAA
ACGAGACTGTCCAACCCTCAAATCAAAAAACTAATAGAGGGGGGACTT
AGCGCCCCCCAAACCATAACACCCATAACAGATTCTCTTAGTGAGGCC
GAATTAGAATGCTTACTTTCTATTCCTCTGGCTCGCAGCCGTCCCTCC
GTGGCTGTATACCTGTCTGGCCCTTGGCTGCAGCCCTCTCAGAATCAA
GCCCTCATGCTTGTGGACACCGGGGCTGAAAATACGGTTCTCCCACAA
AATTGGCTGGTTCGGGATTACCCACGGATCCCCGCCGCAGTGCTCGGA
GCAGGGGGAGTCTCCCGGAACAGATACAATTGGCTACAAGGCCCTCTG
ACCCTGGCTCTAAAACCAGAGGGTCCCTTTATCACCATCCCAAAAATT
TTAGTTGACACTTTCGACAAATGGCAAATTTTAGGACGGGACGTCCTC
TCCCGCCTACAGGCCTCTATCTCCATACCTGAGGAAGTACGCCCCCCT
GTGGTAGGCGTCTTGGATGCCCCCCCGAGCCACATTGGATTAGAACAT
CTGCCCCCTCCACCTGAAGTGCCTCAATTCCCTTTAAACTAGAACGCC
TCCAGGCCCTTCAAGACCTGGTCCATCGCTCTCTGGAGGCAGGTTATA
TCTCCCCCTGGGACGGGCCAGGCAATAATCCAGTCTTCCCGGTACGGA
AACCAAATGGCGCCTGGAGGTTTGTGCATGACCTACGAGCTACAAATG
CTCTTACAAAGCCCATTCCGGCACTCTCCCCCGGACCGCCAGACCTTA
CCGCTATCCCTACACACCTTCCACATATCATTTGCCTAGATCTCAAAG
ACGCCTTCTTCCAGATTCCAGTCGAAGACCGCTTCCGCTCCTACTTTG
CTTTTACCCTCCCTACCCCCGGGGGACTCCAACCTCATAGACGCTTTG
CCTGGCGGGTCCTACCTCAAGGCTTCATTAACAGCCCAGCTCTTTTCG
AACGAGCACTACAGGAACCTCTTCGCCAAGTTTCCGCCGCCTTTTCCC
AGTCTCTTCTGGTGTCCTATATGGACGATATCCTTATCGCTTCGCCTA
CAGAAGAACAGCGGTCACAATGTTATCAAGCCCTGGCTGCCCGCCTCC
GGGACCTAGGGTTTCAGGTGGCATCCGAAAAGACTCGCCAGACGCCTT
CGCCCGTCCCCTTCTTGGGACAAATGGTCCATGAGCAGATTGTCACCT
ACCAGTCCCTACCTACCTTGCAGATCTCATCCCCAATTTCTCTTCACC
AATTACAGGCGGTCTTAGGAGACCTCCAATGGGTCTCTAGGGGCACAC
CCACTACCCGCCGGCCCCTGCAACTTCTCTACTCTTCCCTTAAAGGCA
TCGATGACCCTAGGGCCATCATCCAGCTTTCCCCGGAACAGCTGCAAG
GCATTGCAGAGCTTCGACAAGCCCTGTCCCATAACGCAAGATCTAGAT
ATAACGAGCAAGAACCCCTGCTAGCCTACGTACACCTAACCCGGGCGG
GGTCCACCCTGGTACTCTTCCAAAAGGGCGCTCAATTTCCCCTGGCCT
ACTTCCAGACCCCCTTGACTGACAACCAAGCCTCACCTTGGGGCCTCC
TTCTCCTGCTGGGATGCCAATACCTGCAGACTCAGGCCTTAAGCTCGT
ATGCCAAGCCCATACTTAAATATTATCACAATCTTCCTAAAACTTCTC
TAGACAATTGGATTCAATCATCTGAGGACCCTCGAGTCCAGGAGTTGC
TGCAATTGTGGCCCCAGATTTCCTCTCAGGGAATACAGCCCCCGGGCC
CTTGGAAGACCTTAATCACCAGGGCAGAGGTTTTTTTGACGCCCCAGT
TCTCCCCTGATCCGATTCCTGCGGCCCTTTGCCTCTTTAGTGACGGGG
CTAAAGGACGAGGAGCATATTGCTTGTGGAAGGACCACCTTTTAGACT
TTCAGGCCGTTCCGGCTCCAGAATCCGCTCAAAAGGGAGAACTAGCAG
GTCTCTTGGCGGGCTTAGCAGCCGCCCCGCCTGAACCTGTAAATATAT
GGGTAGATTCCAAATACCTGTACTCTTTGCTCAGAACCCTAGTTCTGG
GAGCTTGGCTTCAACCTGACCCCGTACCCTCCTACGCCCTCCTATATA
AAAGCCTCCTCCGACATCCAGCAATCTTTGTTGGCCATGTCCGGAGCC
ACTCTTCAGCATCCCACCCTATTGCTTCCCTGAACAATTATGTAGATC
AACTGCTTCCCTTAGAAACTCCAGAGCAATGGCATAAGCTCACCCACT
GCAACTCTCGGGCCTTGTCTCGATGGCCGAACCCACGTATCTCTGCCT
GGGACCCCCGTTCCCCCGCTACGCTGTGTGAAACCTGTCAAAAGCTTA
ATCCAACTGGAGGAGGAAAGATGCGAACTATTCAGAGAGGGTGGGCCC
CGAACCATATTTGGCAGGCCGATATAACCCATTATAAATACAAACAGT
TCACCTACGCTCTGCATGTGTTTGTAGATACTTACTCTGGAGCTACTC
ATGCCTCGGCGAAGCGTGGGCTCACCACTCAAATGACCATTGAGGGCC
TTCTTGAGGCCATAGTGCATCTGGGTCGTCCAAAAAAGCTAAACACTG
ACCAAGGTGCAAACTACACCTCCAAAACCTTTGTCAGGTTTTGCCAGC
AGTTCGGAGTTTCCCTTTCTCATCATGTTCCCTACAACCCCACAAGTT
CGGGGTTAGTAGAACGGACAAATGGACTGCTCAAACTTCTTCTATCTA
AATATCACCTAGACGAACCCCACCTTCCCATGACTCAGGCCCTTTCTC
GAGCCCTCTGGACTCACAATCAGATTAACCTCCTACCAATTCTAAAGA
CCAGATGGGAGCTACACCATTCACCCCCACTTGCTGTCATTTCAGAGG
GCGGAGAAACACCCAAGGGCTCTGATAAACTCTTTTTGTACAAGCTCC
CCGGGCAAAACAATCGTCGGTGGCTAGGACCACTCCCGGCCCTAGTCG
AAGCCTCGGGAGGCGCTCTCCTGGCTACTGACCCCCCCGTGTGGGTTC
CCTGGCGTTTGCTGAAAGCCTTCAAATGCCTAAAGAACGACGGTCCCG
AAGACGCCCACAACCGATCATCAGATGGGTAAGTCTCACTCTCACTCT
CCTCGCTCTCTCTCGGCCCATCCAGACTTGGAGATGCTCCCTGTCCCT
AGGAAACCAACAATGGATGACAGCATATAACCAAGAGGCAAAATTTTC
CATCTCCATTGACCAAATACTAGAGGCTCATAATCAGTCACCTTTCTG
TGCCAAGTCTCCCAGATACACCTTGGACTCTGTAAATGGCTATCCTAA
GATCTACTGGCCCCCCCCACAAGGGCGGCGCCGGTTTGGAGCCAGGGC
CATGGTCACATATGATTGCGAGCCCCGATGCCCTTATGTGGGGGCAGA
TCGCTTCGACTGCCCCCACTGGGACAATGCCTCCCAGGCCGATCAAGG
ATCCTTTTATGTCAATCATCAGATTTTATTCCTGCATCTCAAACAATG
TCATGGAATTTTCACTTTAACCTGGGAGATATGGGGATATGATCCCCT
GATCACCTTTTCTTTACATAAGATCCCTGATCCCCCTCAACCCGACTT
TCCCCAGTTGAACAGTGACTGGGTTCCCTCTGTCAGATCATGGGCCCT
GCTTTTAAATCAAACAGCACGGGCCTTCCCAGACTGTGCTATATGTTG
GGAACCTTCCCCTCCCTGGGCTCCCGAAATATTAGTATATAACAAAAC
CATCTCCAGCTCTGGACCCGGCCTCGCCCTCCCGGACGCCCAAATCTT
CTGGGTCAACACGTCCTCGTTTAACACCACCCAAGGATGGCACCACCC
TTCCCAGAGGTTGTTGTTCAATGTTTCTCAAGGCAACGCCTTGTTATT
ACCTCCTATCTCCCTGGTTAATCTCTCTACGGCTTCCTCCGCCCCTCC
TACCCGGGTCAGACGTAGTCCCGTCGCAGCCCTGACCTTAGGCCTAGC
CCTGTCAGTGGGGCTCACTGGAATTAATGTGGCCGTGTCTGCCCTTAG
CCATCAGAGACTCACCTCCCTGATCCACGTTCTGGAGCAAGATCAGCA
ACGCTTGATCACAGCAATTAACCAGACCCACTATAATTTGCTTAATGT
GGCCTCTGTGGTTGCCCAGAACCGACGGGGGCTTGATTGGTTATACAT
CCGGCTGGGTTTTCAAAGCCTATGTCCCACAATTAATGAGCCTTGCTG
TTTCCTGCGCATTCAAAATGACTCCATTATCCGCCTCGGTGATCTCCA
GCCTCTCTCGCAAAGAGTCTCTACAGACTGGCAGTGGCCCTGGAATTG
GGATCTGGGGCTCACTGCCTGGGTGCGAGAAACCATTCATTCTGTTCT
AAGCCTGTTCCTATTAGCCCTTTTTTTGCTCTTCCTGGCCCCCTGCCT
GATAAAATGCTTGACCTCTCGCCTTTTAAAGCTCCTCCGGCAGGCTCC
CCACTTCCCTGAAATCTCCTTAACCCCTAAACCCGATTCTGATTATCA
GGCCTTGCTACCATCTGCACCAGAGATCTACTCTCACCTCTCCCCCGC
CAAACCCGATTACATCAACCTCCGACCCTGTCCTTGATACCCCCGCGT
TTCACGCACCCCCAGGCTGTGGTGGTGCACTGGCTTAGTGGAATAGTC
AGTGTACCATCACAAGCCTCTTCTTGCTGCCGGCACCGAGTTCGAACA
CAGCCCTACCCTGAGCCTCTCTGAGTGCATGACTGAGTGTAGCGCAGA
GAGATTGTCGCTTCTGCGTGTCGCTCAGTCATTTTTTATAGCCGATTG
GGGTTCGCGCCCTTCCGTTGCCTGTGACACAGATAAGACCTCTCTCAC
TTCTGCTTCACCATCCCCCTGCCAGCGTTGGTCTAGTGGAAAGAACTA
ACGCTGACGGGGGCGATTTCTTGCAGCTGTGCTAGCGGGAGGCTCTGG
TGCTGGGGATAAGGTGTGGCCCTTAGCACCACAGTCTCTGCGCCTTTT
GGGTTCGAATCTTCCCCACGCAGCTTCCGCTTTTTACGCCCTGTTGCA
CACCCTTTCTAGAGATACCTGAAAATCTCAGCTCGCACCCTGAGGAAG
GTTGTGGCTCAGAGGTTAAAATAGCTCGGGCCGCAACCTCCCTTTCTT
TTTATTCCACCCTCGCAAGGCCCCGGGTTCTGAGCCCCCTAACGGAGG
TTCAAAATTTCCTCTACTAGGGGATGCTCGGGTCCAAGTGTGCACAAT
ATCTCTTCCAAAAGGTCCTGATGAACGTCTTCCCATGTAACAAGCCCC
AGCAGAGACATTCCAGCCACATCCAGCAGCATTTGGGCCGCCTTTTCT
AACAGTGCCCATAAAGTCCCTTCCGTTTCCACAACGGCTGCCTCTGCT
TCTTCTATTTCCACCTCGGCACCGACTCCCCCGCCGAGCCCTTCGAGC
TCTTCGGGATCCATTACCTGATAACGACAAAATTATTTCTTGTCTTTT
AAGCAAGTGTTGTTGGTTGGGGGCCCCACTCTCTACATGCCTGCCCGG
CCCTGGTTTTGTCCAATGATGTCACCATCGATGCCTGGTGCCCCCTCT
GCGGGCCCCATGAGCGACTCCAATTCGAAAGGATCGACACCACGCTCA
CCTGCGAGACCCACCGTATCAACTGGACCGCCGATGGACGACCTTGCG
GCCTCAATGGAACGTTGTTCCCTCGACTGCATGTCTCCGAGACCCGCC
CCCAAGGGCCCCGACGACTCTGGATCAACTGCCCCCTTCCGGCCGTTC
GCGCTCAGCCCGGCCCGGTTTCACTTTCCCCCTTCGAGCGGTCCCCCT
TCCAGCCCTACCAATGCCAATTGCCCTCGGCCTCTAGCGACGGTTGCC
CCATTATCGGGCACGGCCTTCTTCCCTGGAACAACTTAGTAACGCATC
CTGTCCTCGGAAAAGTCCTTATATTAAATCAAATGGCCAATTTTTCCT
TACTCCCCTCCTTCGATACCCTCCTTGTGGACCCCCTCCGGCTGTCCG
TCTTTGCCCCAGACACCAGGGGAGCCATACGTTATCTCTCCACCCTTT
TGACGCTATGCCCAGCTACTTGTATTCTACCCCTAGGCGAGCCCTTCT
CTCCTAATGTCCCCATATGCCGCTTTCCCCGGGACTCTAATGAACCCC
CCCTTTCAGAATTCGAGCTGCCCCTTATCCAAACGCCCGGCCTGTCCT
GGTCTGTCCCCGCGATCGACCTATTTCTAACCGGTCCCCCTTCCCCAT
GCGACCGGTTACACGTATGGTCCAGTCCTCAGGCCTTACAGCGCTTCC
TTCATGACCCTACGCTAACCTGGTCCGAATTGGTTGCTAGCAGAAAAA
TAAGACTTGATTCCCCCTTAAAATTACAACTGCTAGAAAATGAATGGC
TCTCCCGCCTTTTTTGAGGGGGAGTCATTTGTATGAAAGATCATGCCG
ACCTAGGCGCCGCCACCGCCCCGTAAACCAGACAGAGACGTCAGCTGC
CAGAAAAGCTGGTGACGGCAGCTGGTGGCTAGAATCCCCGTACCTCCC
CAACTTCCCCTTTCCCGAAAAATCCACACCCTGAGCTGCTGACCTCAC
CTGCTGATAAACTAATAAAATGCCGGCCCTGTCGAGTTAGCGGCACCA
GAAGCGTTCTTCTCCTGAGACCCTCGTGCTCAGCTCTCGGTCCTGAGC
TCTCTTGCTCCCGAGACCTTCTGGTCGGCTATCCGGCAGCGGTCAGGT
AAGGCAAACCACGGTTTGGAGGGTGGTTCTCGGCTGAGACCACCGCGA
GCTCTATCTCCGGTCCTCTGACCGTCTCCACGTGGACTCTCTCCTTTG
CCTCCTGACCCCGCGCTCCAAGGGCGTCTGGCTTGCACCCGCGTTTGT
TTCCTGTCTTACTTTCTGTTTCTCGCGGCCCGCGCTCTCTCCTTCGGC
GCCCACTAGCGGCCAGGAGAGACCGGCAAACA

According to the present invention, there is provided a bovine leukemia virus (BLV) in which at least a part of the function of a pol gene is deficient. In the present invention, the sentence that “at least a part of the function of a pol gene is deficient” means that at least a part of the function of the pol gene is deficient in comparison with the wild-type BLV, and specifically means that at least a part of the function of a reverse transcriptase and/or an integrase is deficient. That is, the virus of the present invention does not express the reverse transcriptase and/or the integrase, or, even if it expresses these enzymes, the enzymes do not function normally. The virus does not replicate in an infected subject. For this reason, the virus of the present invention is sometimes referred to as “non-replicating BLV” in the present specification.

Whether or not at least a part of the function of the pol gene is deficient may be determined by preparing a BLV in which at least a part of the pol gene is mutated, and determining whether or not the prepared virus expresses proteins encoded by the pol gene or whether or not the proteins normally function due to the presence or absence of the enzymatic activities of the proteins. The presence or absence of the expression of the proteins can be determined, for example, by a known method such as an immunological assay using an antibody against each of the reverse transcriptase and integrase encoded by the pol gene. The presence or absence of the enzymatic activities of the proteins can be determined, for example, by a known method such as a method for measuring reverse transcriptase activity using colorimetric analysis or a method for measuring integrase activity using the strand transfer ability as an index.

The virus of the present invention is a virus in which at least a part of a pol gene is mutated. In the present invention, the sentence that “at least a part of a pol gene is mutated” means that at least a part of the base sequence of the pol gene is mutated in comparison with the wild-type BLV, and means a mutation that brings a state where at least a part of the function of the pol gene is deficient. The mutation of the pol gene means a mutation in which a deletion, a substitution, an insertion and/or an addition occur/occurs in at least a part of the base sequence of the pol gene, resulting in a state where at least a part of the function of the pol gene is deficient. The function of the pol gene that is deficient in the virus of the present invention is either or both of the reverse transcriptase function and the integrase function, and is preferably the reverse transcriptase function. That is, the virus of the present invention can be said to be a BLV in which a reverse transcriptase region (RT) and/or an integrase region (IN) encoded by the pol gene are/is mutated, or a BLV having a pol gene in which the reverse transcriptase region (RT) and/or the integrase region (IN) are/is mutated. Typical examples of the virus of the present invention include BLVs having a pol gene in which a part of RT (e.g., 1 to 1643 bases, 1 to 1600 bases, 1 to 1500 bases, 1 to 1400 bases, 1 to 1300 bases, 1 to 1200 bases, 1 to 1100 bases, 1 to 1000 bases, 1 to 900 bases, 1 to 850 bases, 1 to 800 bases, 10 to 750 bases, 20 to 700 bases, or 40 to 650 bases) and/or a part of IN (e.g., 1 to 894 bases, 1 to 850 bases, 1 to 800 bases, 10 to 750 bases, 20 to 700 bases, or 40 to 650 bases) is deleted. The deletion of bases in a part of RT and/or IN may be a deletion of continuous bases or a deletion of discontinuous bases.

In the virus of the present invention, the mutation of a part of the base sequence of the pol gene is not limited as long as at least a part of the function of the pol gene is deficient. When the pol gene is a wild-type pol gene of SEQ ID NO: 2 (Table 2), the mutation of RT can be, for example, a mutation in 1^{st} to 1643^{rd}, 1^{st} to 1600^{th}, 1^{st} to 1500^{th}, 1^{st} to 1400^{th}, 1^{st} to 1300^{th}, 1^{st} to 1200^{th}, 1^{st} to 1100^{th}, 1^{st} to 1000^{th}, 10^{th} to 900^{th}, 20^{th} to 800^{th}, 30^{th} to 700^{th}, or 40^{th} to 650^{th} bases from the 5′ side of the base sequence of the pol gene as shown in SEQ ID NO: 2 in Table 2, and the mutation of IN can be, for example, a mutation in 1644^{th} to 2537^{th}, 1650^{th} to 2500^{th}, 1700^{th} to 2400^{th}, 1800^{th} to 2300^{th}, 1700^{th} to 2200^{th}, 1650^{th} to 2100^{th}, 1650^{th} to 2000^{th}, 1650^{th} to 1900^{th}, or 1650^{th} to 1800^{th} bases from the 5′ side of the base sequence of the pol gene as shown in SEQ ID NO: 2. When the pol gene is composed of a base sequence other than that of the wild-type pol gene as shown in SEQ ID NO: 2 (Table 2), the mutation can be a mutation in bases corresponding to the bases of SEQ ID NO: 2. For example, the mutation site can be identified by appropriately aligning the base sequence of the pol gene to be mutated with the base sequence of SEQ ID NO: 2. That is, the mutation site can be identified by appropriately aligning the base sequence of the pol gene to be mutated with the base sequence of SEQ ID NO: 2 using a publicly available homology search software or program such as BLAST (Basic local alignment search tool) (Altschul et al., J. Mol. Biol. 215:403-410 (1990)), FASTA (Peasron et al., Methods in Enzymology 183:63-69 (1990)), or Smith-Waterman (Meth. Enzym., 164, 765 (1988)).

The base sequence of the pol gene of SEQ ID NO: 2 in Table 2 is shown in the 5′ to 3′ direction and corresponds to the underlined base sequence in SEQ ID NO: 1 in Table 1. The number of mutations of RT or the number of mutations of IN is not particularly limited as long as at least a part of the function of the pol gene is deficient. The lower limit value of the number of the mutations can be, for example, 1, 10, 20, 30, 40, 50, 60, 70, 80, or 90, and the upper limit value of the number of the mutations can be 1,000, 900, 800, 700, 600, or 500. These lower limit values and upper limit values can be combined arbitrarily, and the numerical range thereof can be, for example, 1 to 1,000, 10 to 900, 20 to 800, 30 to 700, or 40 to 600.

In the virus of the present invention, the mutation of the base sequence of the pol gene may be a mutation spanning RT and IN. When the pol gene is the wild-type pol gene of SEQ ID NO: 2 (Table 2), such a mutation can be, for example, a mutation in 1 ^{st} to 2537^{th}, 10^{th} to 2500^{th}, 20^{th} to 2400^{th}, 30^{th} to 2300^{th}, 40^{th} to 2200^{th}, 50^{th} to 2100^{th}, 60^{th} to 2000^{th}, 70^{th} to 1900^{th}, 80^{th} to 1850^{th}, 90^{th} to 1800^{th}, 100^{th} to 1750^{th}, 200^{th} to 1700^{th}, 300^{th} to 1650^{th}, 400^{th} to 1650^{th}, 500^{th} to 1650^{th}, 600^{th} to 1650^{th}, 700^{th} to 1650^{th}, 800^{th} to 1650^{th}, 900^{th} to 1650^{th}, or 1000^{th} to 1650^{th} bases from the 5′ side of the base sequence of the pol gene as shown in SEQ ID NO: 2 in Table 2. When the pol gene is composed of a base sequence other than that of the wild-type pol gene as shown in SEQ ID NO: 2 (Table 2), the mutation can be a mutation in bases corresponding to the bases of SEQ ID NO: 2. For example, the mutation site can be identified by appropriately aligning the base sequence of the pol gene to be mutated with the base sequence of SEQ ID NO: 2, as described above.

The number of the mutations spanning RT and IN is not particularly limited as long as at least a part of the function of the pol gene is deficient. The lower limit value of the number of the mutations can be, for example, 1, 10, 20, 30, 40, 50, 60, 70, 80, 90, 100, 200, 300, 400 or 500. The upper limit value of the number of the mutations can be, for example, 2,200, 2,000, 1,800, 1,600, 1,400, 1,200, 1,000, 900, 800, 700, 600 or 500. These lower limit values and upper limit values can be combined arbitrarily, and the numerical range thereof can be, for example, 1 to 2,200, 10 to 2,000, 20 to 1,800, 30 to 1,600, 40 to 1,400, 50 to 1,200, 60 to 1,000, 70 to 900, 80 to 800, 90 to 700, or 100 to 600.

TABLE 2
pol Gene of wild-type BLV (SEQ ID NO: 2)

AACTAGAACGCCTCCAGGCCCTTCAAGACCTGGTCCATCGCTCTCTGGA
GGCAGGTTATATCTCCCCCTGGGACGGGCCAGGCAATAATCCAGTCTTC
CCGGTACGGAAACCAAATGGCGCCTGGAGGTTTGTGCATGACCTACGAG
CTACAAATGCTCTTACAAAGCCCATTCCGGCACTCTCCCCCGGACCGCC
AGACCTTACCGCTATCCCTACACACCTTCCACATATCATTTGCCTAGAT
CTCAAAGACGCCTTCTTCCAGATTCCAGTCGAAGACCGCTTCCGCTCCT
ACTTTGCTTTTACCCTCCCTACCCCCGGGGGACTCCAACCTCATAGACG
CTTTGCCTGGCGGGTCCTACCTCAAGGCTTCATTAACAGCCCAGCTCTT
TTCGAACGAGCACTACAGGAACCTCTTCGCCAAGTTTCCGCCGCCTTTT
CCCAGTCTCTTCTGGTGTCCTATATGGACGATATCCTTATCGCTTCGCC
TACAGAAGAACAGCGGTCACAATGTTATCAAGCCCTGGCTGCCCGCCTC
CGGGACCTAGGGTTTCAGGTGGCATCCGAAAAGACTCGCCAGACGCCTT
CGCCCGTCCCCTTCTTGGGACAAATGGTCCATGAGCAGATTGTCACCTA
CCAGTCCCTACCTACCTTGCAGATCTCATCCCCAATTTCTCTTCACCAA
TTACAGGCGGTCTTAGGAGACCTCCAATGGGTCTCTAGGGGCACACCCA
CTACCCGCCGGCCCCTGCAACTTCTCTACTCTTCCCTTAAAGGCATCGA
TGACCCTAGGGCCATCATCCAGCTTTCCCCGGAACAGCTGCAAGGCATT
GCAGAGCTTCGACAAGCCCTGTCCCATAACGCAAGATCTAGATATAACG
AGCAAGAACCCCTGCTAGCCTACGTACACCTAACCCGGGCGGGGTCCAC
CCTGGTACTCTTCCAAAAGGGCGCTCAATTTCCCCTGGCCTACTTCCAG
ACCCCCTTGACTGACAACCAAGCCTCACCTTGGGGCCTCCTTCTCCTGC
TGGGATGCCAATACCTGCAGACTCAGGCCTTAAGCTCGTATGCCAAGCC
CATACTTAAATATTATCACAATCTTCCTAAAACTTCTCTAGACAATTGG
ATTCAATCATCTGAGGACCCTCGAGTCCAGGAGTTGCTGCAATTGTGGC
CCCAGATTTCCTCTCAGGGAATACAGCCCCCGGGCCCTTGGAAGACCTT
AATCACCAGGGCAGAGGTTTTTTTGACGCCCCAGTTCTCCCCTGATCCG
ATTCCTGCGGCCCTTTGCCTCTTTAGTGACGGGGCTAAAGGACGAGGAG
CATATTGCTTGTGGAAGGACCACCTTTTAGACTTTCAGGCCGTTCCGGC
TCCAGAATCCGCTCAAAAGGGAGAACTAGCAGGTCTCTTGGCGGGCTTA
GCAGCCGCCCCGCCTGAACCTGTAAATATATGGGTAGATTCCAAATACC
TGTACTCTTTGCTCAGAACCCTAGTTCTGGGAGCTTGGCTTCAACCTGA
CCCCGTACCCTCCTACGCCCTCCTATATAAAAGCCTCCTCCGACATCCA
GCAATCTTTGTTGGCCATGTCCGGAGCCACTCTTCAGCATCCCACCCTA
TTGCTTCCCTGAACAATTATGTAGATCAACTGCTTCCCTTAGAAACTCC
AGAGCAATGGCATAAGCTCACCCACTGCAACTCTCGGGCCTTGTCTCGA
TGGCCGAACCCACGTATCTCTGCCTGGGACCCCCGTTCCCCCGCTACGC
TGTGTGAAACCTGTCAAAAGCTTAATCCAACTGGAGGAGGAAAGATGCG
AACTATTCAGAGAGGGTGGGCCCCGAACCATATTTGGCAGGCCGATATA
ACCCATTATAAATACAAACAGTTCACCTACGCTCTGCATGTGTTTGTAG
ATACTTACTCTGGAGCTACTCATGCCTCGGCGAAGCGTGGGCTCACCAC
TCAAATGACCATTGAGGGCCTTCTTGAGGCCATAGTGCATCTGGGTCGT
CCAAAAAAGCTAAACACTGACCAAGGTGCAAACTACACCTCCAAAACCT
TTGTCAGGTTTTGCCAGCAGTTCGGAGTTTCCCTTTCTCATCATGTTCC
CTACAACCCCACAAGTTCGGGGTTAGTAGAACGGACAAATGGACTGCTC
AAACTTCTTCTATCTAAATATCACCTAGACGAACCCCACCTTCCCATGA
CTCAGGCCCTTTCTCGAGCCCTCTGGACTCACAATCAGATTAACCTCCT
ACCAATTCTAAAGACCAGATGGGAGCTACACCATTCACCCCCACTTGCT
GTCATTTCAGAGGGCGGAGAAACACCCAAGGGCTCTGATAAACTCTTTT
TGTACAAGCTCCCCGGGCAAAACAATCGTCGGTGGCTAGGACCACTCCC
GGCCCTAGTCGAAGCCTCGGGAGGCGCTCTCCTGGCTACTGACCCCCCC
GTGTGGGTTCCCTGGCGTTTGCTGAAAGCCTTCAAATGCCTAAAGAACG
ACGGTCCCGAAGACGCCCACAACCGATCATCAGATGGG

The virus of the present invention may have a region other than the pol gene as it is, in the full length of the sequence between the 5′ LTR and 3′ LTR of the wild-type BLV as shown in SEQ ID NO: 1, as long as at least a part of the function of the pol gene is deficient and the functions of the BLV genes other than the pol gene are retained. Alternatively, the virus may have a sequence having 80% or more (preferably 85% or more, more preferably 90% or more, even more preferably 95% or more, and particularly preferably 96% or more, 97% or more, 98% or more, 99% or more, 99.5% or more, or 99.8% or more) identity to that of the region (wild type) other than the pol gene. The term “identity” as used herein refers to the degree of identity, for example, when sequences to be compared are appropriately aligned, and means an appearance rate (%) of exactly matched amino acids between the sequences. In the calculation of the identity, for example, the presence of gaps in the sequence and the nature of amino acids are taken into consideration (Wilbur, Natl. Acad. Sci. U.S.A. 80:726-730 (1983)). The alignment can be performed, for example, by using an arbitrary algorithm. Specifically, publicly available homology search software such as BLAST (Basic local alignment search tool) (Altschul et al., J. Mol. Biol. 215:403-410 (1990)), FASTA (Peasron et al., Methods in Enzymology 183:63-69 (1990)), or Smith-Waterman (Meth. Enzym., 164, 765 (1988)) can be used. The identity can be calculated, for example, using the publicly available homology search program as described above, and can be calculated, for example, using a default parameter in the homology algorithm BLAST (https://blast.ncbi.nlm.nih.gov/Blast.cgi) of the National Center for Biotechnique Information (NCBI).

The virus of the present invention can be obtained by allowing a cell having a BLV gene in which at least a part of the function of the pol gene is deficient to produce it. The origin of the cell is not limited, and the cell may be a naturally occurring cell, a cell obtained by self-cloning, or a cell prepared by a gene recombination technique. However, from the viewpoint that the virus is used as a vaccine, a naturally occurring cell or a cell obtained by self-cloning can be preferably used.

### Cell and Method for Producing Same

According to the present invention, there is also provided a cell comprising a gene of a bovine leukemia virus (BLV) in which at least a part of the function of a pol gene is deficient. The phrase “comprising a gene” as used herein means that the gene is contained in the cell in an expressible manner, and typically means that the gene has been introduced into the cell and is in a transformed state. The cell of the present invention may be a naturally occurring cell, a cell obtained by self-cloning, or a cell prepared by a gene recombination technique, as long as the cell comprises a polynucleotide encoding a BLV in which at least a part of the function of the pol gene is deficient. However, from the viewpoint that the virus is used as a vaccine, a naturally occurring cell or a cell obtained by self-cloning can be preferably used.

According to another aspect of the present invention, there is provided a method for producing the cell of the present invention. When the cell of the present invention is produced by a gene recombination technique, it can be prepared, for example, by introducing into a host cell an expression vector having incorporated thereinto a polynucleotide encoding a BLV in which at least a part of the function of the pol gene is deficient. To make the function of the BLV pol gene deficient, the entire or a part of RT and/or IN encoded by the pol gene may be deleted. Also, the pol gene may be mutated by substitution, deletion, insertion and/or addition of bases to prevent expression of a normal reverse transcriptase or integrase. Alternatively, a foreign gene may be inserted into RT or IN. The deletion or mutation of a gene and the insertion of a foreign gene can be carried out by known homologous recombination or site-directed mutagenesis.

The host cell for producing the BLV of the present invention is not particularly limited as long as it is capable of transcription and translation of an expression vector. Examples of the host cell include insect cells (e.g., silkworm cells), amphibian cells, reptilian cells, avian cells, fish cells, and mammalian cells (e.g., PK15 cells, HEK293 cells, HeLa cells, COS cells, BHK cells, CHL cells, and CHO cells). The host cell is preferably a mammalian cell.

The transformation method of the host cell may be selected depending on the type of host cell or the like, and the transformation can be performed, for example, by an electroporation method, a lithium acetate method, a calcium phosphate method, a lipofection method, a particle gun method, or the like.

The cell of the invention can be implemented according to the descriptions of the virus of the invention, in addition to the above description.

### Method for Producing Virus

According to the present invention, there is also provided a method for producing a virus. The method for producing a virus according to the present invention comprises the step of culturing the cell of the present invention. The method for producing a virus according to the present invention may further comprise the step of recovering a virus produced by the cell of the present invention. The recovered virus may be purified as necessary. The purification method may be appropriately selected from known methods depending on the virus to be produced. In the case of a virus that is accumulated within the host cell, the virus may be purified after disruption of the host cell. In the case of a virus that is released outside the host cell, the virus can be purified and recovered, for example, from a culture supernatant of the host cell. The method for producing a virus according to the present invention can be implemented according to the descriptions of the virus of the present invention, and the cell of the present invention and the method for producing the same, in addition to the above description.

### BLV Vaccine

According to the present invention, there is also provided a BLV vaccine. The vaccine of the present invention is characterized by comprising the virus of the present invention. That is, the vaccine of the present invention can be used to prevent infection with a BLV or to treat (or ameliorate symptoms of) EBL, because administration of the vaccine into a subject induces an immune response against the BLV (for example, production of a specific antibody or proliferation of a cytotoxic T cell) and promotes production of a neutralizing antibody against the BLV and production of an interferon or the like in the subject.

The vicus of the present invention, which is contained in the vaccine of the present invention, preferably has at least a gene encoding a structural protein, more preferably has at least any one of p24 of the gag gene and gp51 and gp30 of the env gene, and even more preferably has at least gp51, from the viewpoint of effectively inducing an immune response against the BLV in the subject to be administered with the vaccine. Without being bound by the following theory, it is believed that, in the infection with the BLV, when gp51 binds to a receptor on the surface of the cell membrane of the host (subject), the virus and the cell membrane of the host fuse by virtue of the membrane fusion activity of gp30, thereby allowing entry of the core of the BLV into the host cell (Bai L, et al., FASEB Journal, 2019, fj201901528R).

In the vaccine of the present invention, the content of the virus of the present invention can be appropriately set depending on the subject to be administered with the vaccine, the administration method, and the form of administration. For example, when the subject is cattle, the content can be 0.01 mg to 10 mg (preferably 0.5 mg to 5 mg) per cattle. The vaccine of the present invention may be administered once, twice, three or more times depending on the subject, and the number of administrations may be added whenever the antibody titer decreases in the subject.

The vaccine of the present invention can comprise a pharmaceutically acceptable carrier, a lubricant, a preservative, a stabilizer, a wetting agent, an emulsifier, a salt for adjusting an osmotic pressure, a buffer, a colorant, an antioxidant, a viscosity adjusting agent, an activator (a concept which also includes carbonate apatite, sodium hydroxide, alum, an incomplete/complete Freund's adjuvant and the like, as immunostimulants), nanoparticles or the like. Examples of the pharmaceutically acceptable carrier include water, various salt solutions, alcohols, vegetable oils, and mineral oils.

Examples of the form of the vaccine of the present invention include an injection, a liquid medicine, a suspension, an emulsion, a powder, a granule, and a capsule. The form of administration of the vaccine may be oral administration or parenteral administration (subcutaneous administration, transnasal administration, intraperitoneal administration, intracapsular administration, intramuscular administration, or an intravenous administration).

The vaccine of the present invention can be used in the prevention or treatment of bovine leukosis. According to another aspect of the present invention, there is provided a method for preventing or treating bovine leukosis, comprising the step of administering the vaccine of the present invention to a subject.

In the present invention, the “subject” is a non-human animal or a non-human mammal, and examples thereof include mammals such as cattle (Bos taurus), zebu (Bos indicus), water buffalo (Bubalus bubalis), sheep, goat, pig, mouse, rat, rabbit, cat, and monkey. Examples of the cattle include dairy breeds, meat breeds, dual-purpose breeds for dairy and meat, draft breeds, and dual-purpose breeds for draft and meat. Specific examples of the cattle include Japanese cattle such as Japanese Black cattle and Japanese Shorthorn cattle, Holstein cattle, Jersey cattle, and local breeds of various countries. From the viewpoint of preventing or treating bovine leukosis, the subject is preferably cattle, zebu and water buffalo, more preferably cattle and water buffalo, and even more preferably cattle.

The vaccine of the present invention can be implemented according to the descriptions of the virus of the present invention and the method for producing the same, and the cell of the present invention and the method for producing the same, in addition to the above description.

### EXAMPLES

The present invention will be described in more detail by way of the following examples, but is not limited to these examples.

### Example 1: Establishment of Non-Replicating BLV-Producing Cell Line

The present inventors previously constructed a CMVΔU3-pBLV-IF plasmid having a high virus production amount of BLV as disclosed in JP 2019-24351 A, but constructed a CMVΔU3-pBLV-416 plasmid using, as a based plasmid, a pBLV-416 plasmid cloned as a wild-type strain having a higher virus production amount than that of the CMVΔU3-pBLV-IF plasmid. The CMVΔU3-pBLV-416 plasmid was prepared in the same manner as in the preparation of the CMVΔU3-pBLV-IF plasmid described in JP 2019-24351 A. This CMVΔU3-pBLV-416 plasmid was introduced into PK15 cells to attempt to establish a cell line having a high BLV production amount. As a result of repeating cell subculture and seven runs of cloning, the inventors succeeded in creating a CMVΔU3-pBLV-416 plasmid-introduced mutated stable cell line (sometimes referred to as “PK15-BLVΔRT cell line” in the present specification) in which the virus produced had lost its replicating ability and integrating ability while retaining infectivity. As a result of analysis of the entire base sequence of the provirus integrated into this cell line, it was confirmed that 46 bp to 696 bp in the RT region of the pol gene were deleted (see FIG. 1).

### Example 2: Study (1) Using pBLV-416ΔRT-Introduced Cell

In Example 2, a CMVΔU3-pBLV-416ΔRT plasmid introduced with a deletion of 46 bp to 696 bp in the RT region of the pol gene was prepared, and viral proteins produced from cells (pBLV-416ΔRT-introduced cells) introduced with this molecular clone were analyzed.

### (1) Method

a. Plasmid

The CMVΔU3-pBLV-416 plasmid was prepared in the same manner as in the preparation of the CMVΔU3-pBLV-IF plasmid described in JP 2019-24351 A. CMVΔU3-pBLV416ART was prepared by site-directed mutagenesis using the CMVΔU3-pBLV-416 plasmid as a template (Bai L, et al., Retrovirology, 2015; 12(1):106, Inabe K, et al., J. Virol., 1999; 73: 1293-1301., Matsuura R, et al., 2019; 11(12):1140). The deletion of the RT region of the pol gene in pBLV-416ΔRT was confirmed by PCR.

b. Cell

African green monkey kidney-derived cells (COS-1) were used as the cells into which the plasmid was to be introduced. The COS-1 cells were maintained on a DMEM medium added with 10% fetal bovine serum (FBS) and 1×Penicillin-Streptomycin-Glutamine (PSG) at 37°° C. in a CO₂ incubator.

c. Transfection

The COS-1 cells (5×10⁵ cells/60 mm-dish) were subjected to transfection using the reagents and plasmids indicated in Tables 3 to 5, and the cells and culture supernatants were recovered 48 hours after transfection. In the present specification, the cells subjected to transfection are sometimes referred to as “introduced cells.”

TABLE 3
Transfection of pBLV-416ΔRT

| | | |
|---|---|---|
| FuGENE HD | 32 | μL |
| pBLV-416ΔRT | 8 | μg |
| Opti-MEM | 400 | μL |

TABLE 4
Transfection of pBLV-416

| | | |
|---|---|---|
| FuGENE HD | 32 | μL |
| pBLV-416 | 8 | μg |
| Opti-MEM | 400 | μL |

TABLE 5
Transfection of pBluescript II KS(−)

| | | |
|---|---|---|
| FuGENE HD | 32 | μL |
| pBluescript II KS(−) | 8 | μg |
| Opti-MEM | 400 | μL |

d. Western Blotting

The expression of the viral proteins was confirmed by Western blotting using a BLV-infected bovine serum or a non-BLV-infected bovine serum.

### (2) Result

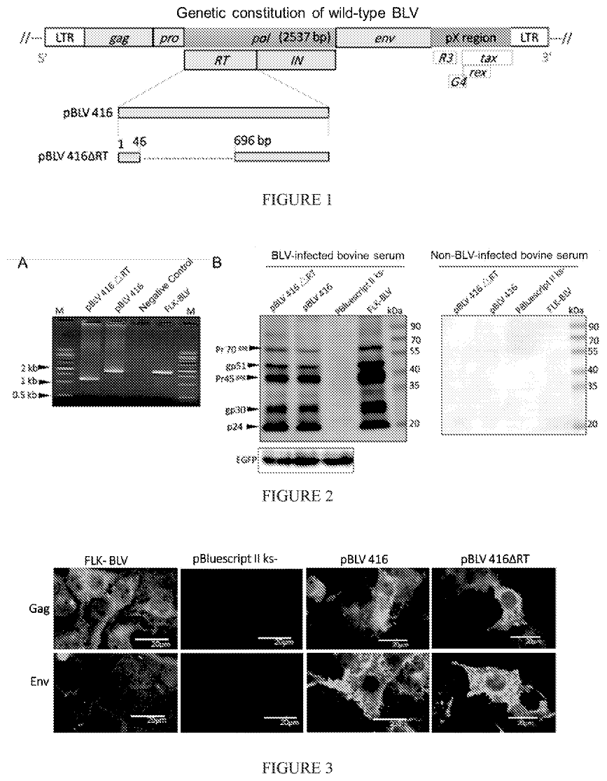

The results were as shown in FIG. 2. As a result of amplifying the pol gene region by PCR, it was confirmed that, in the pBLV-416ΔRT plasmid, a fragment shorter by 650 bp than that in the pBLV-416 was amplified, and that a region corresponding to 46 bp to 696 bp in the RT region of the pol gene was deleted (FIG. 2A). As a result of the Western blotting, it was confirmed that, in the case of using the BLV-infected bovine serum, the expression levels of structural proteins such as Gag protein (p24) and Env proteins (gp51 and gp30) were not affected by the deletion of the pol gene in the pBLV-416ΔRT-introduced cells as compared with the pBLV-416-introduced cells (FIG. 2B, left), whereas no viral protein was detected in the case of using the non-BLV-infected bovine serum (FIG. 2B, right).

### Example 3: Study (2) Using pBLV-416ΔRT-Introduced Cell

In Example 3, the influences of the deletion of the pol gene on the intracellular localization and expression levels of the viral proteins were studied using pBLV-416ΔRT-introduced cells.

### (1) Method

a. Plasmid

A plasmid to similar that in Example 2 (1) a was used.

b. Cell

COS-1 cells were used similarly as in Example 2 (1) b.

c. Western Blotting

The viral proteins were confirmed by Western blotting using an anti-Gag antibody and an anti-Env antibody.

d. Immunofluorescent Antibody Method

The intracellular localization of the viral proteins was confirmed by an immunofluorescent antibody method using an anti-Gag antibody and an anti-Env antibody.

### (2) Result

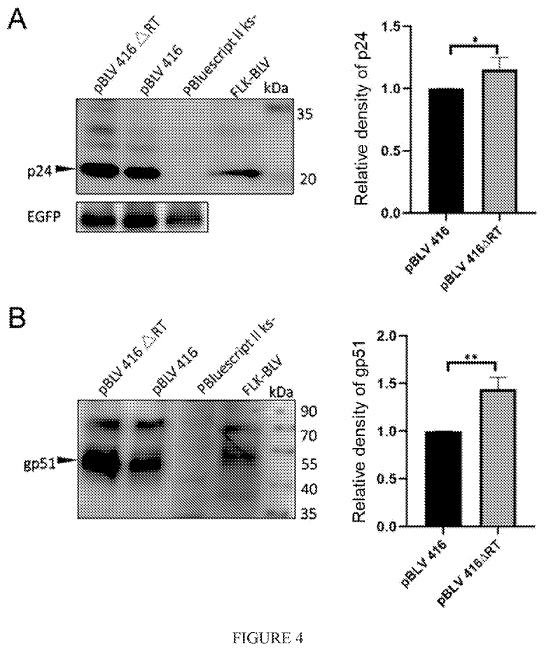

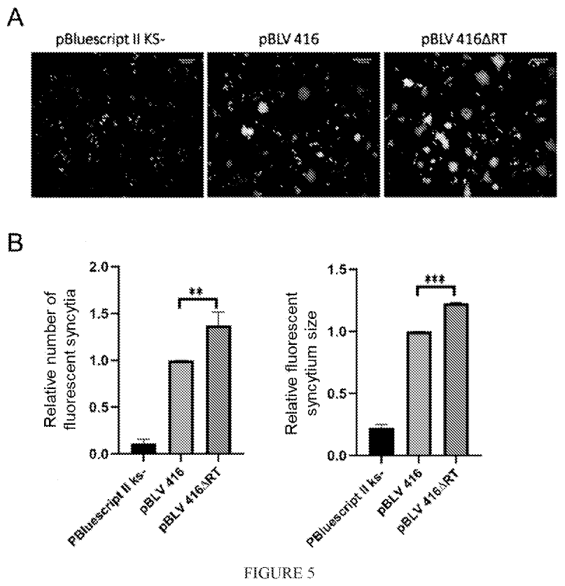

The results were as shown in FIGS. 3 and 4. In the pBLV-416ΔRT-introduced cells, as compared with the pBLV-416-introduced cells, the influence of the deletion of the pol gene on the intracellular localization of the Gag protein (p24) and the Env protein (gp51) was not observed (FIG. 3), but increases in expression levels of both p24 and gp51 were confirmed (FIGS. 4A and 4B).

### Example 4: Study (3) Using pBLV-416ΔRT-Introduced Cell

In Example 4, the syncytium formation ability and cell-to-cell infection ability of the virus produced from the pBLV-416ΔRT-introduced cells were studied.

### (1) Method

a. Syncytium Formation Ability

The pBLV-416ΔRT plasmid or pBLV-416 plasmid was introduced into 293T cells together with a pEGFP-N1 plasmid (EGFP expression plasmid) to evaluate the syncytium formation ability. The negative control used was 293T cells into which a pBluescript II KS(−) plasmid, as an empty vector, was introduced together with an EGFP-N1 plasmid.

b. Evaluation of Cell-to-Cell Infection Ability

To evaluate the cell-to-cell infection ability, the pBLV-416ΔRT plasmid or pBLV-416 plasmid was introduced into COS-1 cells, which were cocultured with CC81-GREMG cells (reporter cells for the BLV). The CC81-GREMG cells express EGFP in a BLV Tax-dependent manner. When the BLV-producing cells and the CC81-GREMG cells fuse due to the Env protein of the BLV to form syncytia, syncytia having expressed EGFP are observed. Therefore, the cell-to-cell infection ability can be evaluated.

### (2) Result

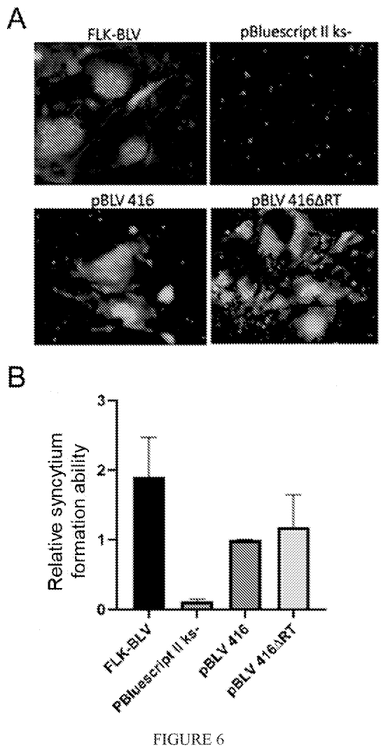

The results were as shown in FIGS. 5 and 6. It was confirmed that both the pBLV-416ΔRT-introduced cells and the pBLV-416-introduced cells formed syncytia (FIG. 5A). It was confirmed that the number and size of syncytia were larger in the pBLV-416ΔRT-introduced cells than those in the pBLV-416-introduced cells (FIG. 5B). The results indicated that the deletion of the pol gene enhances the syncytium formation ability in the pBLV-416ΔRT-introduced cells.

Regarding the cell-to-cell infection ability, it was also confirmed that the number and size of syncytia formed were larger in the pBLV-416ΔRT-introduced cells than those in the pBLV-416-introduced cells (FIGS. 6A and B). The results suggested the possibility that the deletion of the pol gene may enhance the cell-to-cell infection.

### Example 5: Study (4) Using pBLV-416ΔRT-Introduced Cell

In Example 5, the production amount of the virus produced from the pBLV-416ΔRT-infected cells and the reverse transcriptase activity were analyzed.

### (1) Method

a. Plasmid

A plasmid to similar that in Example 2 (1) a was used.

b. Cell

COS-1 cells were used similarly as in Example 2 (1) b.

c. Western Blotting

The viral proteins were detected by Western blotting using an anti-Gag antibody and an anti-Env antibody.

d. Virus Release Amount

To confirm the virus production amounts of the pBLV-416ΔRT-introduced cells and the pBLV-416-introduced cells, the amount of p24 in the culture supernatant was measured by Capture ELISA. Specific procedures are as follows. A BLV-positive serum was diluted with a carbonate buffer solution adjusted to a pH of 9.0 and immobilized on a 96-well plate. Next, the culture supernatant of the pBLV-416ΔRT-introduced cells or the pBLV-416-introduced cells and Tween 20 were added to the plate on which the serum was immobilized to capture p24. The amount of captured p24 was then measured by a colorimetric method using an anti-BLV p24 antibody (BLV3, VMRD) and an HRP-labeled anti-mouse IgG antibody.

e. Reverse Transcriptase Activity

The virus released into the culture supernatant was recovered by ultracentrifugation, and the reverse transcriptase activity in the viral particles was measured using a Reverse Transcriptase Assay, Colorimetric kit (Roche).

f. Viral Infectivity

To evaluate the viral infectivity, the virus-containing culture supernatant was added to CC81-GREMG cells to confirm the formation of syncytia. Using 2 μg of the virus, p24 and gp51 in the virus were quantified by Western blotting.

### (2) Result

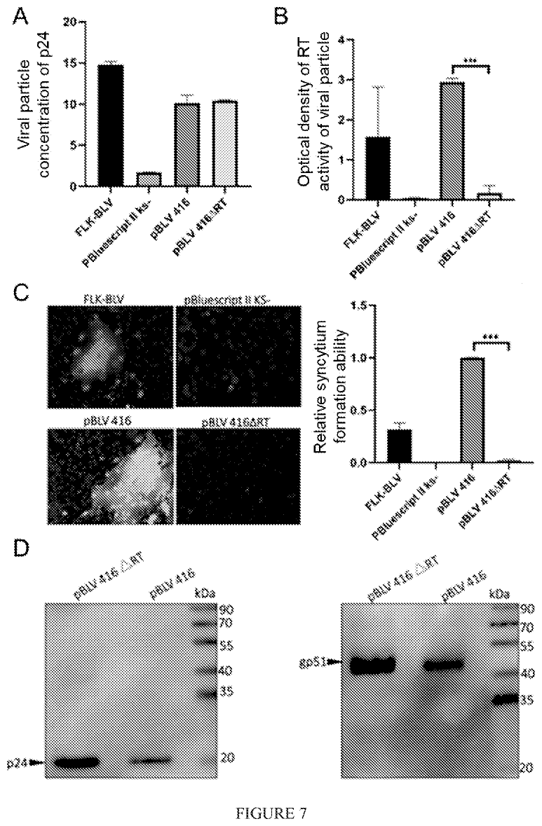

The results were as shown in FIG. 7. There was no difference in virus release amount due to the deletion of the pol gene between the pBLV-416ΔRT-introduced cells and the pBLV-416-introduced cells (FIG. 7A). Furthermore, the virus released from the pBLV-416ΔRT-introduced cells did not exhibit reverse transcriptase activity (FIG. 7B), nor did it form syncytia (FIG. 7C). Furthermore, the Gag protein (p24) and the Env protein (gp51) were detected in the pBLV-416ΔRT-introduced cells, indicating that the deletion of the pol gene does not affect viral maturation (FIG. 7D).

### Example 6: Study Using pBLV-IFΔRT-Introduced Cell

In Example 6, a CMVΔU3-pBLV-IFΔRT plasmid introduced with a deletion of 46 bp to 696 bp in the RT region of the pol gene was prepared, and viral protein produced from cells (pBLV-IFΔRT-introduced cells) introduced with this molecular clone were analyzed.

### (1) Method

a. Plasmid

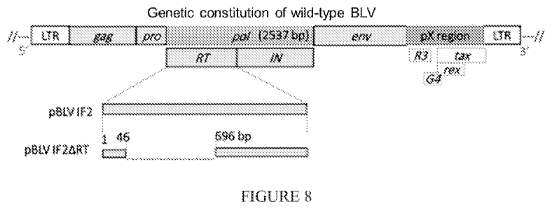

A CMVΔU3-pBLV-IF2 plasmid was prepared according to the description of JP 2019-24351 A. Similarly as in Example 2 (1) a, a CMVΔU3-pBLV-IF2ΔRT plasmid was prepared by site-directed mutagenesis using the CMVΔU3-pBLV-IF plasmid as a template (Tajima S, et al., J Virol. 2000; 74 (23):10939-10949) (FIG. 8).

b. Cell

COS-1 cells and 293T cells were used as the cells into which the plasmid was to be introduced.

c. Western Blotting

The viral proteins were confirmed by Western blotting using an anti-Gag antibody and an anti-Env antibody.

d. Syncytium Formation Ability

The same procedures as in Example 4 (1) a were conducted.

### (2) Result

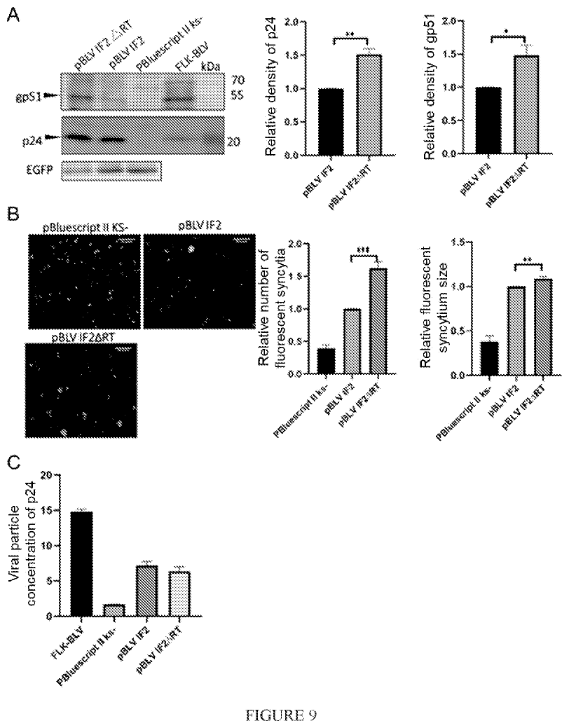

The results were as shown in FIG. 9. It was confirmed that the expression levels of the Gag protein (p24) and the Env protein (gp51) increased in the pBLV-IF2ΔRT-introduced cells (293T cells) as compared with the pBLV-IF2-introduced cells (293T cells) (FIG. 9A). Also, it was confirmed that the number and size of syncytia increased in the pBLV-IF2ΔRT-introduced cells as compared with the pBLV-IF2-introduced cells (FIG. 9B).

In the pBLV-IF2ΔRT-introduced cells (COS-1 cells), an equivalent amount of Gag protein (p24) to that in the pBLV-IF2-introduced cells (COS-1 cells) was detected by Capture ELISA, and no influence of the deletion of the pol gene was observed (FIG. 9C).

### Example 7: Study on Bovine Leukosis Vaccine

In Example 7, the virus produced from the pBLV-416ΔRT-introduced cells was inoculated into mice to study the effect of the virus as a vaccine.

### (1) Method

a. Plasmid

A plasmid to similar that in Example 1 (1) a was used.

b. Cell

COS-1 cells were used as the cells into which the plasmid was to be introduced.

c. Virus

The culture supernatant of the pBLV-416ΔRT-introduced cells or the pBLV-416-introduced cells was centrifuged at 3,000 rpm for 10 minutes to remove cell debris, and then ultracentrifuged at 141,118×g and 4° C. for 2 hours to remove the culture supernatant. Then, the precipitate was resuspended in PBS to purify the virus.

d. Inoculation Into Mouse

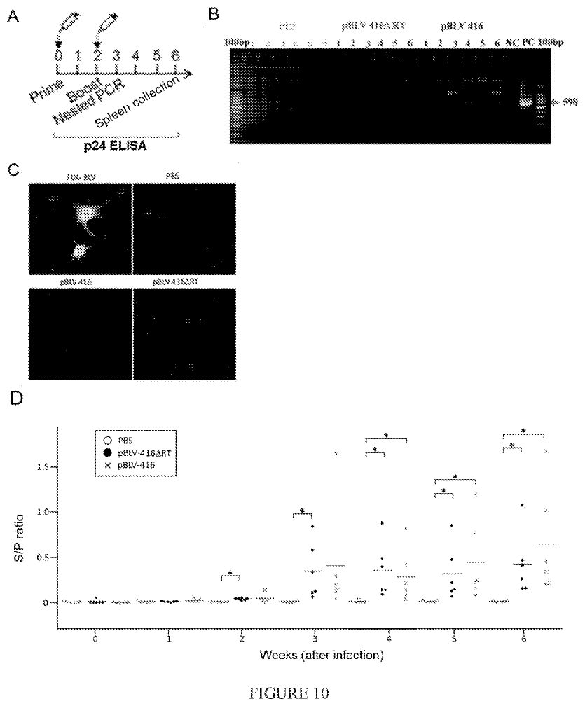

According to the schedule shown in FIG. 10A, 100 μg of the virus was inoculated into mice in the 0^{th} and 2^{nd} weeks, and PBS was inoculated as the negative control (n=6 per group). Blood sampling was conducted from 0 to 6 weeks after inoculation. Following euthanasia 6 weeks after inoculation, the spleen was extracted. To confirm viral proliferation within the mouse bodies, the BLV genes in the blood cells 3 weeks after inoculation were detected using a Nested PCR method. In addition, spleen cells were co-cultured with CC81-GREMG cells to evaluate the syncytium formation ability. In addition, anti-p24 antibodies from 0 to 6 weeks after inoculation were quantified by ELISA.

### (2) Result

The results were as shown in FIG. 10. In the mice inoculated with the virus produced by the pBLV-416ΔRT-introduced cells, no BLV gene was detected (FIG. 10B), and no syncytium was observed even when the spleen cells extracted from the mice were co-cultured with CC81-GREMG (FIG. 10C). On the other hand, it was confirmed that the anti-p24 antibody increased over time in the mice inoculated with the virus produced by the pBLV-416ΔRT-introduced cells (FIG. 10D). These results suggested the possibility that the virus produced from the pBLV-416ΔRT-introduced cells may be useful as a non-replicating BLV vaccine.

### Example 8: Study (1) Using Non-Replicating BLV-Producing Cell Line

In Example 8, the entire base sequence of the provirus integrated into the PK15-BLVΔRT cell line successfully created in Example 1 was analyzed again. As a result, it was confirmed that 43 bp to 696 bp in the RT region of the pol gene were deleted (see FIG. 11). The results showed a difference of three bases in number of bases deleted in the RT region of the pol gene from the analysis results in Example 1. This difference is considered to be due to addition of further mutations during subculture after establishment of the PK15-BLVΔRT cell line. In addition, as compared with the base sequence of the wild-type BLV, a mutation of two bases and a deletion of one base were confirmed after 696 bp in the pol gene, but these mutations did not affect the protein expression.

### Example 9: Study (2) Using Non-Replicating BLV-Producing Cell Line

In Example 9, the expression and localization of the viral proteins in cells, and the amount of virus released were studied using the PK15-BLVΔRT cell line.

### (1) Method

a. Western Blotting

The viral proteins were confirmed by Western blotting using an anti-Gag antibody and an anti-Env antibody.

b. Immunofluorescent Antibody Method

The intracellular localization of the viral proteins was confirmed by the immunofluorescent antibody method using an anti-Gag antibody and an anti-Env antibody.

c. Virus Release Amount

The virus release amount of the PK15-BLVΔRT cell line was measured by the Capture ELISA method. Specific procedures are as follows. The culture supernatant of the cells was centrifuged at 141,118×g for 2 hours to remove the supernatant. The precipitate was then resuspended in an amount of PBS one-hundredth the amount of the culture supernatant to concentrate the virus, and the concentration of p24 was measured. The concentration of viral p24 was calculated from a standard curve created using, as a standard, His-p24 antigen expressed using E. coli and purified (FIG. 14B).

### (2) Result

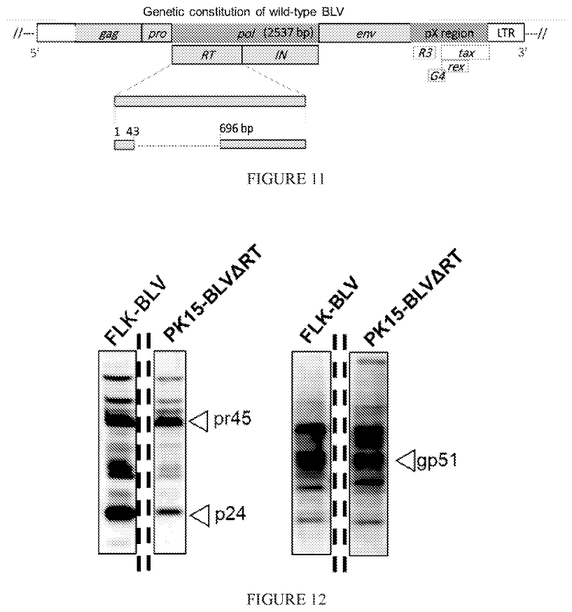

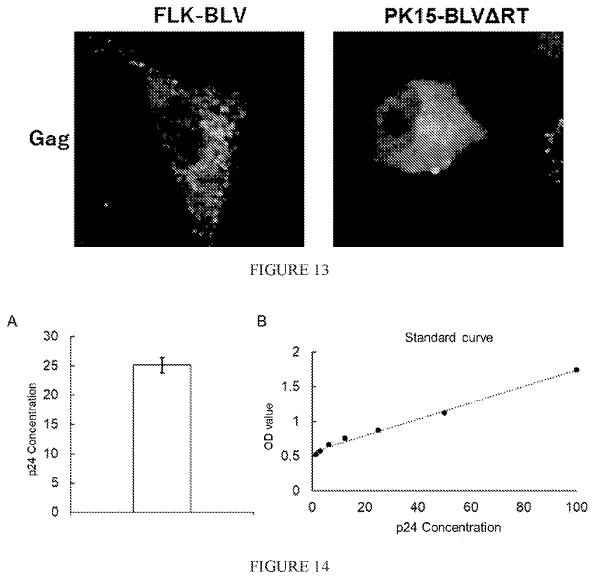

The results were as shown in FIGS. 12 to 14. The results in FIG. 12 confirmed that the PK15-BLVΔRT cell line expressed the viral structural proteins p24 and gp51. Further, as compared with the positive control, persistently BLV-infected cell FLK-BLV, no changes in molecular weights of p24 and gp51 due to the deletion of the pol gene were observed. From the results in FIG. 13, spotted accumulation was observed within the cells, similarly as in the positive control, persistently BLV-infected cell FLK-BLV. The results confirmed that the deletion of the pol gene does not affect the intracellular localization of p24. From the results in FIG. 14A, the concentration of p24 was 25.1±1.2 μg, confirming that a sufficient amount of virus was released from the PK15-BLVΔRT cell line. These results suggested the possibility that the virus produced from the PL15-BLVΔRT cell line may be useful as a non-replicating BLV vaccine.

1. A bovine leukemia virus (BLV) in which at least a part of the function of a pol gene is deficient.

2. The virus according to claim 1, wherein the function of the pol gene is the function of a reverse transcriptase and/or an integrase.

3. The virus according to claim 1, wherein at least a part of the pol gene is mutated.

4. The virus according to claim 3, wherein the mutation is a deletion of 1 to 1643 bases in a reverse transcriptase region of the pol gene and/or 1 to 894 bases in an integrase region of the pol gene.

5. The virus according to claim 1, which does not replicate in an infected subject.

6. A non-replicating bovine leukemia virus (BLV)-producing cell, comprising a gene of a BLV in which at least a part of the function of a pol gene is deficient.

7. A method for producing a non-replicating BLV, comprising the step of culturing the cell according to claim 6.

8. A method for preventing or treating enzootic bovine leukosis (EBL), comprising the step of inoculating a subject excluding a human with the virus according to claim 1.

9. A method for producing a non-replicating bovine leukemia virus (BLV)-producing cell, comprising the step of introducing, into a host cell, a gene of a BLV in which at least a part of the function of a pol gene is deficient.


SEQUENCE LISTING (2 sequences)

SEQ ID NO: 1 - DNA, 8720 bp, Bovine leukemia virus
  source 1..8720: mol_type = other DNA
tgtatgaaagatcatgccgacctaggcgccgccaccgccccgtaaaccagacagagacgtcagctgccagaaaagctggtgacggcagctggtggctagaatccccgtacctccccaacttcccctttcccgaaaaatccacaccctgagctgctgacctcacctgctgataaactaataaaatgccggccctgtcgagttagcggcaccagaagcgttcttctcctgagaccctcgtgctcagctctcggtcctgagctctcttgctcccgagaccttctggtcggctatccggcagcggtcaggtaaggcaaaccacggtttggagggtggttctcggctgagaccaccgcgagctctatctccggtcctctgaccgtctccacgtggactctctcctttgcctcctgaccccgcgctccaagggcgtctggcttgcacccgcgtttgtttcctgtcttactttctgtttctcgcggcccgcgctctctccttcggcgcccactagcggccaggagagaccggcaaacaattgggggctcgtccgggattgatcaccccggaaccctaacaactctctggacccaccccctcggcggcattttgggtctctccttcaaattatatcatgggaaattccccctcctataacccccccgctggtatctccccctcagactggctcaaccttctgcaaagcgcgcaaaggctcaatccgcgaccctctcctagcgattttaccgatttaaagaattacatccattggtttcataagacccagaaaaaaccatggactttcacttctggtggccccacctcatgtccacccgggaggttcggccgggttccccttgtcttggccaccctaaacgaagtgctctcaaacgaagggggcgccccgggtgcatcggccccagaagaacaaccccccccttatgacccccccgccgttttgccaatcatatctgaagggaatcgcaaccgccatcgtgcttgggcactccgagaattacaagatatcaaaaaggaaattgaaaataaggcaccgggttcgcaagtatggatacaaacactacgacttgcaatcctgcaggccgaccctactccggctgacctagaacaactttgccaatatattgcttccccggtcgaccaaacggcccatatgaccagcctaacggcagcaattgccgccgctgaagcggccaacaccctccagggttttaacccccaaaacgggaccctaacccaacaatcagctcagcccaacgccggggatcttagaagtcaatatcaaaacctctggcttcaggcctggaaaaatctccctactcgtcctttagtacaaccttggtccaccatcgtccaaggccccgccgaaagctatgtagagtttgtcaaccggttacaaatttcattagctgacaaccttcccgacggagtccctaaggaacccattattgactcccttagttatgcaaatgctaacaaagagtgtcagcaaattttgcaagggcgaggcctagtggccgccccggtggggcaaaaactgcaggcttgcgcacattgggcccccaagatgaaacagcctgcaattctcgtccacaccccagggcccaagatgcccgggcctcggcaaccggcccccaaaaggcctcccccaggaccatgctatcgatgcctcaaagaaggccattgggcccgggattgtcctaccaaggccaccggcccccctccgggaccttgccccatatgtaaagatccttcccattggaaacgagactgtccaaccctcaaatcaaaaaactaatagaggggggacttagcgccccccaaaccataacacccataacagattctcttagtgaggccgaattagaatgcttactttctattcctctggctcgcagccgtccctccgtggctgtatacctgtctggcccttggctgcagccctctcagaatcaagccctcatgcttgtggacaccggggctgaaaatacggttctcccacaaaattggctggttcgggattacccacggatccccgccgcagtgctcggagcagggggagtctcccggaacagatacaattggctacaaggccctctgaccctggctctaaaaccagagggtccctttatcaccatcccaaaaattttagttgacactttcgacaaatggcaaattttaggacgggacgtcctctcccgcctacaggcctctatctccatacctgaggaagtacgcccccctgtggtaggcgtcttggatgcccccccgagccacattggattagaacatctgccccctccacctgaagtgcctcaattccctttaaactagaacgcctccaggcccttcaagacctggtccatcgctctctggaggcaggttatatctccccctgggacgggccaggcaataatccagtcttcccggtacggaaaccaaatggcgcctggaggtttgtgcatgacctacgagctacaaatgctcttacaaagcccattccggcactctcccccggaccgccagaccttaccgctatccctacacaccttccacatatcatttgcctagatctcaaagacgccttcttccagattccagtcgaagaccgcttccgctcctactttgcttttaccctccctacccccgggggactccaacctcatagacgctttgcctggcgggtcctacctcaaggcttcattaacagcccagctcttttcgaacgagcactacaggaacctcttcgccaagtttccgccgccttttcccagtctcttctggtgtcctatatggacgatatccttatcgcttcgcctacagaagaacagcggtcacaatgttatcaagccctggctgcccgcctccgggacctagggtttcaggtggcatccgaaaagactcgccagacgccttcgcccgtccccttcttgggacaaatggtccatgagcagattgtcacctaccagtccctacctaccttgcagatctcatccccaatttctcttcaccaattacaggcggtcttaggagacctccaatgggtctctaggggcacacccactacccgccggcccctgcaacttctctactcttcccttaaaggcatcgatgaccctagggccatcatccagctttccccggaacagctgcaaggcattgcagagcttcgacaagccctgtcccataacgcaagatctagatataacgagcaagaacccctgctagcctacgtacacctaacccgggcggggtccaccctggtactcttccaaaagggcgctcaatttcccctggcctacttccagacccccttgactgacaaccaagcctcaccttggggcctccttctcctgctgggatgccaatacctgcagactcaggccttaagctcgtatgccaagcccatacttaaatattatcacaatcttcctaaaacttctctagacaattggattcaatcatctgaggaccctcgagtccaggagttgctgcaattgtggccccagatttcctctcagggaatacagcccccgggcccttggaagaccttaatcaccagggcagaggtttttttgacgccccagttctcccctgatccgattcctgcggccctttgcctctttagtgacggggctaaaggacgaggagcatattgcttgtggaaggaccaccttttagactttcaggccgttccggctccagaatccgctcaaaagggagaactagcaggtctcttggcgggcttagcagccgccccgcctgaacctgtaaatatatgggtagattccaaatacctgtactctttgctcagaaccctagttctgggagcttggcttcaacctgaccccgtaccctcctacgccctcctatataaaagcctcctccgacatccagcaatctttgttggccatgtccggagccactcttcagcatcccaccctattgcttccctgaacaattatgtagatcaactgcttcccttagaaactccagagcaatggcataagctcacccactgcaactctcgggccttgtctcgatggccgaacccacgtatctctgcctgggacccccgttcccccgctacgctgtgtgaaacctgtcaaaagcttaatccaactggaggaggaaagatgcgaactattcagagagggtgggccccgaaccatatttggcaggccgatataacccattataaatacaaacagttcacctacgctctgcatgtgtttgtagatacttactctggagctactcatgcctcggcgaagcgtgggctcaccactcaaatgaccattgagggccttcttgaggccatagtgcatctgggtcgtccaaaaaagctaaacactgaccaaggtgcaaactacacctccaaaacctttgtcaggttttgccagcagttcggagtttccctttctcatcatgttccctacaaccccacaagttcggggttagtagaacggacaaatggactgctcaaacttcttctatctaaatatcacctagacgaaccccaccttcccatgactcaggccctttctcgagccctctggactcacaatcagattaacctcctaccaattctaaagaccagatgggagctacaccattcacccccacttgctgtcatttcagagggcggagaaacacccaagggctctgataaactctttttgtacaagctccccgggcaaaacaatcgtcggtggctaggaccactcccggccctagtcgaagcctcgggaggcgctctcctggctactgacccccccgtgtgggttccctggcgtttgctgaaagccttcaaatgcctaaagaacgacggtcccgaagacgcccacaaccgatcatcagatgggtaagtctcactctcactctcctcgctctctctcggcccatccagacttggagatgctccctgtccctaggaaaccaacaatggatgacagcatataaccaagaggcaaaattttccatctccattgaccaaatactagaggctcataatcagtcacctttctgtgccaagtctcccagatacaccttggactctgtaaatggctatcctaagatctactggccccccccacaagggcggcgccggtttggagccagggccatggtcacatatgattgcgagccccgatgcccttatgtgggggcagatcgcttcgactgcccccactgggacaatgcctcccaggccgatcaaggatccttttatgtcaatcatcagattttattcctgcatctcaaacaatgtcatggaattttcactttaacctgggagatatggggatatgatcccctgatcaccttttctttacataagatccctgatccccctcaacccgactttccccagttgaacagtgactgggttccctctgtcagatcatgggccctgcttttaaatcaaacagcacgggccttcccagactgtgctatatgttgggaaccttcccctccctgggctcccgaaatattagtatataacaaaaccatctccagctctggacccggcctcgccctcccggacgcccaaatcttctgggtcaacacgtcctcgtttaacaccacccaaggatggcaccacccttcccagaggttgttgttcaatgtttctcaaggcaacgccttgttattacctcctatctccctggttaatctctctacggcttcctccgcccctcctacccgggtcagacgtagtcccgtcgcagccctgaccttaggcctagccctgtcagtggggctcactggaattaatgtggccgtgtctgcccttagccatcagagactcacctccctgatccacgttctggagcaagatcagcaacgcttgatcacagcaattaaccagacccactataatttgcttaatgtggcctctgtggttgcccagaaccgacgggggcttgattggttatacatccggctgggttttcaaagcctatgtcccacaattaatgagccttgctgtttcctgcgcattcaaaatgactccattatccgcctcggtgatctccagcctctctcgcaaagagtctctacagactggcagtggccctggaattgggatctggggctcactgcctgggtgcgagaaaccattcattctgttctaagcctgttcctattagccctttttttgctcttcctggccccctgcctgataaaatgcttgacctctcgccttttaaagctcctccggcaggctccccacttccctgaaatctccttaacccctaaacccgattctgattatcaggccttgctaccatctgcaccagagatctactctcacctctcccccgccaaacccgattacatcaacctccgaccctgtccttgatacccccgcgtttcacgcacccccaggctgtggtggtgcactggcttagtggaatagtcagtgtaccatcacaagcctcttcttgctgccggcaccgagttcgaacacagccctaccctgagcctctctgagtgcatgactgagtgtagcgcagagagattgtcgcttctgcgtgtcgctcagtcattttttatagccgattggggttcgcgcccttccgttgcctgtgacacagataagacctctctcacttctgcttcaccatccccctgccagcgttggtctagtggaaagaactaacgctgacgggggcgatttcttgcagctgtgctagcgggaggctctggtgctggggataaggtgtggcccttagcaccacagtctctgcgccttttgggttcgaatcttccccacgcagcttccgctttttacgccctgttgcacaccctttctagagatacctgaaaatctcagctcgcaccctgaggaaggttgtggctcagaggttaaaatagctcgggccgcaacctccctttctttttattccaccctcgcaaggccccgggttctgagccccctaacggaggttcaaaatttcctctactaggggatgctcgggtccaagtgtgcacaatatctcttccaaaaggtcctgatgaacgtcttcccatgtaacaagccccagcagagacattccagccacatccagcagcatttgggccgccttttctaacagtgcccataaagtcccttccgtttccacaacggctgcctctgcttcttctatttccacctcggcaccgactcccccgccgagcccttcgagctcttcgggatccattacctgataacgacaaaattatttcttgtcttttaagcaagtgttgttggttgggggccccactctctacatgcctgcccggccctggttttgtccaatgatgtcaccatcgatgcctggtgccccctctgcgggccccatgagcgactccaattcgaaaggatcgacaccacgctcacctgcgagacccaccgtatcaactggaccgccgatggacgaccttgcggcctcaatggaacgttgttccctcgactgcatgtctccgagacccgcccccaagggccccgacgactctggatcaactgcccccttccggccgttcgcgctcagcccggcccggtttcactttcccccttcgagcggtcccccttccagccctaccaatgccaattgccctcggcctctagcgacggttgccccattatcgggcacggccttcttccctggaacaacttagtaacgcatcctgtcctcggaaaagtccttatattaaatcaaatggccaatttttccttactcccctccttcgataccctccttgtggaccccctccggctgtccgtctttgccccagacaccaggggagccatacgttatctctccacccttttgacgctatgcccagctacttgtattctacccctaggcgagcccttctctcctaatgtccccatatgccgctttccccgggactctaatgaaccccccctttcagaattcgagctgccccttatccaaacgcccggcctgtcctggtctgtccccgcgatcgacctatttctaaccggtcccccttccccatgcgaccggttacacgtatggtccagtcctcaggccttacagcgcttccttcatgaccctacgctaacctggtccgaattggttgctagcagaaaaataagacttgattcccccttaaaattacaactgctagaaaatgaatggctctcccgccttttttgagggggagtcatttgtatgaaagatcatgccgacctaggcgccgccaccgccccgtaaaccagacagagacgtcagctgccagaaaagctggtgacggcagctggtggctagaatccccgtacctccccaacttcccctttcccgaaaaatccacaccctgagctgctgacctcacctgctgataaactaataaaatgccggccctgtcgagttagcggcaccagaagcgttcttctcctgagaccctcgtgctcagctctcggtcctgagctctcttgctcccgagaccttctggtcggctatccggcagcggtcaggtaaggcaaaccacggtttggagggtggttctcggctgagaccaccgcgagctctatctccggtcctctgaccgtctccacgtggactctctcctttgcctcctgaccccgcgctccaagggcgtctggcttgcacccgcgtttgtttcctgtcttactttctgtttctcgcggcccgcgctctctccttcggcgcccactagcggccaggagagaccggcaaaca

SEQ ID NO: 2 - DNA, 2537 bp, Bovine leukemia virus
  source 1..2537: mol_type = other DNA
aactagaacgcctccaggcccttcaagacctggtccatcgctctctggaggcaggttatatctccccctgggacgggccaggcaataatccagtcttcccggtacggaaaccaaatggcgcctggaggtttgtgcatgacctacgagctacaaatgctcttacaaagcccattccggcactctcccccggaccgccagaccttaccgctatccctacacaccttccacatatcatttgcctagatctcaaagacgccttcttccagattccagtcgaagaccgcttccgctcctactttgcttttaccctccctacccccgggggactccaacctcatagacgctttgcctggcgggtcctacctcaaggcttcattaacagcccagctcttttcgaacgagcactacaggaacctcttcgccaagtttccgccgccttttcccagtctcttctggtgtcctatatggacgatatccttatcgcttcgcctacagaagaacagcggtcacaatgttatcaagccctggctgcccgcctccgggacctagggtttcaggtggcatccgaaaagactcgccagacgccttcgcccgtccccttcttgggacaaatggtccatgagcagattgtcacctaccagtccctacctaccttgcagatctcatccccaatttctcttcaccaattacaggcggtcttaggagacctccaatgggtctctaggggcacacccactacccgccggcccctgcaacttctctactcttcccttaaaggcatcgatgaccctagggccatcatccagctttccccggaacagctgcaaggcattgcagagcttcgacaagccctgtcccataacgcaagatctagatataacgagcaagaacccctgctagcctacgtacacctaacccgggcggggtccaccctggtactcttccaaaagggcgctcaatttcccctggcctacttccagacccccttgactgacaaccaagcctcaccttggggcctccttctcctgctgggatgccaatacctgcagactcaggccttaagctcgtatgccaagcccatacttaaatattatcacaatcttcctaaaacttctctagacaattggattcaatcatctgaggaccctcgagtccaggagttgctgcaattgtggccccagatttcctctcagggaatacagcccccgggcccttggaagaccttaatcaccagggcagaggtttttttgacgccccagttctcccctgatccgattcctgcggccctttgcctctttagtgacggggctaaaggacgaggagcatattgcttgtggaaggaccaccttttagactttcaggccgttccggctccagaatccgctcaaaagggagaactagcaggtctcttggcgggcttagcagccgccccgcctgaacctgtaaatatatgggtagattccaaatacctgtactctttgctcagaaccctagttctgggagcttggcttcaacctgaccccgtaccctcctacgccctcctatataaaagcctcctccgacatccagcaatctttgttggccatgtccggagccactcttcagcatcccaccctattgcttccctgaacaattatgtagatcaactgcttcccttagaaactccagagcaatggcataagctcacccactgcaactctcgggccttgtctcgatggccgaacccacgtatctctgcctgggacccccgttcccccgctacgctgtgtgaaacctgtcaaaagcttaatccaactggaggaggaaagatgcgaactattcagagagggtgggccccgaaccatatttggcaggccgatataacccattataaatacaaacagttcacctacgctctgcatgtgtttgtagatacttactctggagctactcatgcctcggcgaagcgtgggctcaccactcaaatgaccattgagggccttcttgaggccatagtgcatctgggtcgtccaaaaaagctaaacactgaccaaggtgcaaactacacctccaaaacctttgtcaggttttgccagcagttcggagtttccctttctcatcatgttccctacaaccccacaagttcggggttagtagaacggacaaatggactgctcaaacttcttctatctaaatatcacctagacgaaccccaccttcccatgactcaggccctttctcgagccctctggactcacaatcagattaacctcctaccaattctaaagaccagatgggagctacaccattcacccccacttgctgtcatttcagagggcggagaaacacccaagggctctgataaactctttttgtacaagctccccgggcaaaacaatcgtcggtggctaggaccactcccggccctagtcgaagcctcgggaggcgctctcctggctactgacccccccgtgtgggttccctggcgtttgctgaaagccttcaaatgcctaaagaacgacggtcccgaagacgcccacaaccgatcatcagatggg